(19) United States
(12) Patent Application Publication
(10) Pub. No.: US 2004/0071855 A1
(43) Pub. Date: Apr. 15, 2004

(54) Glucosamine as a food and beverage additive

(76) Inventor: Willem Wassenaar, Toronto (CA)

Correspondence Address: Johnston Avisar Wassenaar, 56 The Esplanade, Suite 401, Toronto, ON, M5E1A7, CA

(21) Appl. No.: 10/273,854

(22) Filed: Oct. 21, 2002

(30) Foreign Application Priority Data
Oct. 15, 2002 (CA) 2,408,023

(51) Int. Cl. A61K 31/7008; A23L 2/00

(52) U.S. Cl. 426/590; 514/62

(57) ABSTRACT

This invention relates to the addition of N-acetylglucosamine to foods and beverages and to the use of N-acetylglucosamine as a sweetening agent. This invention also relates to foods and beverages prepared with N-acetylglucosamine and methods of preparing the same. This invention provides a preferable means of ingesting the desired daily amount of N-acetylglucosamine for its beneficial effects on the body, for example, alleviation of pain and inflammation in a patient suffering from inflammatory bowel disorder or osteoarthritis.

### FIELD OF THE INVENTION

This invention relates to the addition of N-acetylglucosamine to foods and beverages and to the use of N-acetylglucosamine as a sweetening agent. This invention also relates to foods and beverages prepared with N-acetylglucosamine and methods of preparing the same.

### BACKGROUND OF THE INVENTION

Glucosamine and N-acetylglucosamine (1)

Chemistry

Glucosamine is a glucose molecule with an amino group at position 2. The molecular weight of glucosamine is 179.17 and the melting point is 88° C. It is usually sold as a sulphate or chloride salt.

N-acetylglucosamine is a glucosamine that is acetylated on the amino group. The molecular weight is of N-acetylglucosamine is 221 and the melting point is 205° C.

Biological Role of Glucosamine and N-acetylglucosamine

Glucosamine has no specific biological function other than to act as a precursor for N-acetylglucosamine. N-acetylglucosamine is a major constituent of glycoproteins (proteins with a few sugars attached) and proteoglycans (sugar chains with a few proteins attached). Exogenously administered glucosamine and N-acetylglucosamine are phosphorylated in vivo and incorporated into macromolecules such as glycoproteins and proteoglycans. N-acetylglucosamine also has important regulatory functions: a) providing negative feed back to the fructose-6-P glutamine transamidase reaction which is responsible for the production of glucosamine from fructose-6-P and b) inhibiting the production of nitric oxide synthetase, COX-2 and IL-6 in human chondrocytes stimulated with IL-1. Glucosamine on the other hand is not effective in same test systems.

Glycoproteins are proteins to which sugars are covalently bound. Sugars can make up 4% of the molecular mass as in the case of IgG and 82% of the molecular mass as in human gastric glycoproteins. Glycoproteins form the major constituents of mucus secreted by epithelial cells. Glycoproteins are important determinants of cell group behaviours. Glycoproteins determine the ABO typing of red cells and determine cell membrane function. Secreted hormones such as FSH, LH, HCG and erythropoietin are all glycoproteins. Similarly circulating enzymes such as prothrombin are also glycoproteins.

Proteoglycans are carbohydrate chains (glycosaminoglycans) linked covalently to a protein core. Proteins make up very little of the proteoglycan molecule, usually less than 5%, and in one case hyaluronic acid, 0%. Proteoglycans used to be called mucopolysaccharides.

Proteoglycans consist of long unbranched heteropolysaccharide chains made up of repeating units of disaccharide. These repeating units usually consist of one hexosamine (e.g. N-acetylglucosamine) and one uronic acid (e.g. glycuronic acid). The addition of one or more sulphate groups to these repeating units leads to the production of molecules such as heparin.

Hyaluronate (hyaluronic acid) is a co-polymer of N-acetylglucosamine and glucuronic acid. This simple molecular structure can form very long chains with molecular weights of 10⁵ or 10⁷. These large molecular weights with their polyelectrolytic characteristics cause this molecule, when in solution, to occupy a very large volume. This solution is an ideal lubricant and shock absorber. Hyaluronic acid is found predominantly in synovial fluid, vitreous humour and the umbilical cord.

Chondroitin Sulphate

Chondroitin sulphate is the most abundant of the glycosaminoglycans in the human body. Individual polysaccharide chains are attached to the serine amino acid of proteins. In this molecule the repeating disaccharide unit is N-acetylgalactosamine and glucuronic acid. The source of N-acetylgalactosamine is N-acetylglucosamine through the reversible epimerization reaction. The N-acetylgalactosamine molecule in chondroitin is sulphated at position 4 or 6.

Chondroitin disaccharide chains are made up of 30 to 50 repeating disaccharide units. Each protein core may in turn have as many as 100 such chains leading to a molecule with a molecular weight of 2×10⁶. Chondroitin sulphate is a prominent component of cartilage, tendons, ligaments and the aorta. Chondroitin sulphate has also been isolated from brain, kidney and lung.

Biosynthetic steps incorporate exogenously administered Glucosamine and N-Acetylglucosamine into UDP-N-Acetylglucosamine-6-P, the molecular form that is incorporated into glycoproteins, proteoglycans and hyaluronic acid. Glucosamine requires one more synthetic step than N-Acetylglucosamine. This acetylation step can be inhibited by salicylates and ethanol.

Evidence of Utility

In Vitro Studies

Inflammatory conditions can also interfere with the incorporation of glucosamine into glycosaminoglycans. Intestinal mucosal from patients with ulcerative colitis and Crohn's disease and from patients without inflammatory disease were studied in vitro. N-acetylglucosamine is preferentially incorporated into glycoproteins over glucosamine. In normal subjects—ratio ranges were from 0.04 to 0.26 with a mean 0.097. Thus on average 10 times more N-acetylglucosamine is incorporated into macromolecules than glucosamine. In IBD the mean ratio of incorporation of glucosamine to N-acetylglucosamine is 0.039 for Crohn's and 0.031 for ulcerative colitis. In other words, 25 to 32 times more N-acetylglucosamine was incorporated into macromolecules compared to glucosamine (2).

Glucosamine and N-acetylglucosamine inhibit IL-1 P-induced NO production in normal human articular chondrocytes. The effect of the sugars on NO production is specific, since several other monosaccharides, including glucose, glucuronic acid, and N-acetylmannosamine, do not express this activity. Furthermore, N-acetylglucosamine polymers, such as the dimer and the trimer, do not affect NO production. At a 10 mM concentration N-Acetylglucosamine's ability to inhibit IL-1β-induced NO production was 5 times greater than glucosamine's ability to inhibit IL-1β-induced NO production. Both molecules inhibited NO production in a dose dependent manner.

The effect of various sugars on IL-1β induced NO Production

| | Inhibitory Activity |
|---|---|
| Glucose | Nil |
| Glucuronic Acid | Nil |
| Glucosamine | Weak |
| N-Acetylglucosamine | Strong |
| N-Acetylgalactosmine | Strong |
| N-Acetylmannosamine | Nil |

The observed suppression of IL-1β-induced NO production is associated with inhibition of inducible NO synthase mRNA and protein expression. In addition, N-acetylglucosamine also suppresses the production of IL-1β-induced cyclooxygenase-2 and IL-6. The constitutively expressed cyclooxygenase-1, however, was not affected by the sugar. N-acetylglucosamine-mediated inhibition of the IL-1β stimulation of the human chondrocyte was specific and identifies a novel mechanism of inhibition of inflammation in the human joint (3).

Investigators found that N-acetylglucosamine's inhibitory activity occurred at the level of mRNA and protein expression. They also included other markers of inflammation. As summarized in the table below, N-acetylglucosamine effected the expression of IL-6 and messenger RNA for NO synthetase and cyclooxygenase-2 (3).

| | Inducible NO | | | | COX-1 | |
|---|---|---|---|---|---|---|
| | Synthetase Expression | | COX-2 Expression | | Expression | |
| | mRNA | Protein | mRNA | Protein | Protein | IL-6 Production |
| N-Acetylglucosamine | inhibits | inhibits | inhibits | inhibits | No Inhibition | inhibits |

In Vivo Studies —Clinical Evidence

There have been many clinical studies with glucosamine and fewer with N-acetylglucosamine. Many of the glucosamine studies have been small or have lacked the appropriate controls. This is a review of the most significant studies.

Osteoarthritis

One of the most exciting studies supporting the use of glucosamine in osteoarthritis was recently published in the Lancet (“the Reginster trial”). What makes the Reginster trial so important is that glucosamine has structure modifying effects on the osteoarthritic knee joint. Previous studies had shown that glucosamine is able to reduce symptoms such as pain (4). The Reginster randomized double blind placebo controlled trial in 106 patients on placebo and 106 on 1.5 grams of glucosamine sulphate per day was carried out in patients over the age of 50 with primary knee osteoarthritis. The patients on placebo experienced significant joint space narrowing and the patients on glucosamine did not. The results were statistically significant at p<0.05. The incidence and type of adverse events reported over the three-year period was not different between the two groups. Routine laboratory tests did not show any abnormalities in system organs or metabolic functions. Glycaemic homeostasis was similar in both groups. This is the first study to show that glucosamine can have disease-modifying activity (5).

In an earlier study, glucosamine was compared with ibuprofen. In this double-blind trial, 178 Chinese patients were randomized to one of 2 groups. One group received 1,500 mg of glucosamine sulphate and the other group received 1,200 mg of ibuprofen. End points included knee pain at rest, with movement and knee tenderness and swelling. Both treatments reduced symptoms of asteoarthritis over the 4-week course of therapy. Glucosamine was better tolerated. None of the glucosamine treated patients dropped out of the study because of adverse events but 10% of ibuprofen patients dropped out because of side effects (6).

Many other glucosamine trials in osteoarthritis have been reported. Some showed glucosamine to be effective (7, 8), others reported no benefit from glucosamine therapy (9, 10). The positive trials generally had more patients enrolled and thus may have been able to pick up a statistical difference when smaller trials could not. The Reginster trial differed from all others in two respects a) 1,500 mg was given once daily and b) the patients were treated for 3 years. Most other trials had a treatment period of only 4 to 8 weeks.

Osteoarthritis of the Temporomandibular Joint (TMJ)

In one published clinical trial, glucosamine sulphate was compared to ibuprofen in a group of patients with osteoarthritis of the temporomandibular joint. Forty women and five men received either glucosamine sulphate (500 mg tid) or ibuprofen (400 mg tid) for 90 days in a randomized double blind study. End points included TMJ pain with function, pain-free, and voluntary maximum mouth opening and masticatory muscle tenderness. Acetaminophen (500 mg) use for breakthrough pain was also recorded.

Forty-five patients entered the study and thirty-nine patients completed the study (21 GS, 18 ibuprofen). Four discontinued due to stomach upset (3 ibuprofen, one GS), one due to dizziness (GS), and one due to inadequate pain control (ibuprofen). Fifteen GS (71%) and 11 ibuprofen (61%) improved, with positive clinical response taken as a 20% decrease TMJ pain with function. The number of patients with positive clinical response was not statistically different between groups (p=0.73). Between-group comparison revealed that patients taking GS had a significantly greater decrease in TMJ pain with function, and acetaminophen use between Day 90 and 120 compared with patients taking ibuprofen. The investigators also observed a carryover effect in the glucosamine treated group (11).

Inflammatory Bowel Disease

The University Department of Paediatric Gastroenterology at the Royal Free Hospital in London used N-acetylglucosamine in the treatment of inflammatory bowel disease (12). N-acetylglucosamine (total daily dose 3-6 g) was administered orally as adjunct therapy to 12 children with severe treatment-resistant inflammatory bowel disease (10 Crohn's disease, 2 ulcerative colitis). Seven of these children suffered from symptomatic strictures. In addition, similar doses were administered rectally as sole therapy in nine children with distal ulcerative colitis or proctitis resistant to steroids and antibiotics. Eight of the children given oral N-acetylglucosamine showed clear improvement, while four required resection. Of the children with symptomatic Crohn's stricture, only 3 of 7 required surgery over a mean follow-up of >2.5 years, and endoscopic or radiological improvement was detected in the others. Rectal administration induced remission in two cases, clear improvement in three and no effect in two. In all cases biopsied there was evidence of histological improvement. N-acetylglucosamine in this small trial was an effective treatment in chronic inflammatory bowel disease.

Absorption & Excretion

N-acetylglucosamine and glucosamine hydrochloride were administered IV in doses as high as 20 grams per human subject. The half-life of N-acetylglucosamine was 220 minutes and the half-life of glucosamine hydrochloride was 150 minutes. In 24 hours, 57% of the administered glucosamine hydrochloride was excreted in the urine and 54% of the administered N-acetylglucosamine was excreted in the urine. When administered with 7 units of insulin only glucosamine hydrochloride showed a rise in blood sugar. Neither agent showed a rise in blood sugar when administered without insulin. In the five subjects studied there were no adverse events reported (13).

The most extensive investigation of glucosamine administered to humans comes from the work of Setnikar, published in 2000 (14). Healthy male volunteers were administered glucosamine mixed with trace amounts of radioactive glucosamine. Glucosamine was administered by IV, IM and oral routes. Radioactivity was measured in whole plasma and deproteinized plasma. The concentration found in deproteinized plasma was subtracted from the concentration in whole plasma in order to obtain the amount incorporated in plasma proteins. The concentration in deproteinized plasma, which represents free glucosamine, disappears quickly and the amount in plasma proteins increased over time as glucosamine is incorporated into the globulin fraction.

When given by the oral route, about 10% of the administered dose is excreted by the kidneys and 11% is excreted via the feces. Compared with IV administration at 100%, the absolute bioavailability of glucosamine after oral administration is 44%, and the absolute bioavailability of glucosamine after IM administration is 93%.

In a parallel study Setnikar et al also examined the absorption of glucosamine tablets and a glucosamine solution. Based on the urine excretion of glucosamine, glucosamine tablets given TID and glucosamine oral solution given once per day, they considered these two presentations to be bioequivalent (14).

Safety

In the pediatric inflammatory bowel disease trial, patients were give 3 to 6 grams of N-acetylglucosamine in three divided doses daily. The authors reported that in this high dose study “no adverse side-effects of treatment were noted in any patient” attributable to the N-acetylglucosamine.

The three year glucosamine sulphate study in osteoarthritis published in the Lancet is more detailed in its reporting of adverse events. This was a placebo controlled double blind study. Most of the reported symptoms were transient and mild to moderate in severity. Over the three-year treatment period some patients dropped out due to adverse events or were lost to follow-up. This is presented in the table below.

| | | Glucosamine |
|---|---|---|
| Withdrawal from Study | Placebo | Sulphate |
| Due to Adverse Events | 18 | 21 |
| Lost to Follow-up | 12 | 14 |
| Lack of Efficacy | 5 | 3 |
| Totals | 35 | 38 |

Among the adverse events leading to patient's dropout, few single episodes were serious and all were judged as unrelated to the study treatments, mostly because such episodes were attributable to pre-existing or concomitant conditions in this elderly population. There was no statistical difference in adverse events between the placebo and the glucosamine sulphate groups.

Routine laboratory tests did not show any great abnormalities in system organs or metabolic functions in the two groups during the study. There was no change in glycaemic homeostasis with fasting plasma glucose concentrations decreasing slightly in the glucosamine sulphate group.

These two studies lead to the conclusion that this amine sugar has little if any potential for harm during clinical use.

Effect on Insulin and Glucose

The clinical trial results presented above uniformly report that N-acetylglucosamine and glucosamine use does not interfere with glucose utilization. In vitro animal studies suggest that over activity of the hexosamine pathway represents an important mechanism by which hyperglycemia causes insulin resistance. In a recent study published in The Journal of Clinical Endocrinology & Metabolism twenty human volunteers were infused with intravenous glucosamine. The authors reported the following conclusions:

Forearm glucosamine infusion did not affect total body insulin sensitivity.

Baseline arteriovenous glucose differences were low and were not affected by glucosamine infusion.

Glucosamine infusion did not affect forearm skeletal muscle glucose uptake.

Glucosamine infusion did not alter forearm blood flow.

The results of blood chemistry monitoring in the long term clinical trials combined with this human pharmacology study confirm that glucosamine use does not have negative impact on glycaemic control in normals (15).

### SUMMARY OF THE INVENTION

This invention relates to the addition of N-acetylglucosamine to foods and beverages and to the use of N-acetylglucosamine as a sweetening agent. This invention also relates to foods and beverages prepared with N-acetylglucosamine and methods of preparing the same. This invention provides a preferable means of ingesting the desired daily amount of N-acetylglucosamine for its beneficial effects on the body, for example, alleviation of pain and inflammation in a patient suffering from inflammatory bowel disorder or osteoarthritis.

### DETAILED DESCRIPTION OF THE INVENTION

A review of the literature indicates that glucosamine and N-acetylglucosamine have two biological activities that may be responsible for the reported positive clinical results in osteoarthritis and inflammatory bowel disease. In the first mode of action, excess amounts of these sugars may drive enzymatic activity to increase the production of hyaluronic acid and chondroitin sulphate in inflamed joints. This may serve to increase lubrication and the rebuilding of cartilage. The second possible mode of action is the decreased production of inflammatory cytokines by N-acetylglucosamine. The direct anti-inflammatory activity of N-acetylglucosamine has only recently been elucidated.

There is clear published evidence that glucosamine is effective in osteoarthritis. There is also evidence that N-acetylglucosamine is effective in inflammatory bowel disease. Glucosamine and N-acetylglucosamine have been given to people for periods up to three years without serious adverse effects.

The side effects observed in various clinical trials with N-acetylglucosamine and glucosamine are few and mild. In the case of glucosamine sulphate or glucosamine hydrochloride the side effects may in part be due to the salt portion of the preparation. The salt portion of the molecule may make up as much as 40% of the administered dose. Another source of side effects could be due to the excipients in the numerous commercial preparations on the market.

The molecular form of glucosamine can have an important bearing on how much glucosamine is actually ingested. Most commercially available glucosamine come as either the sulphate or the hydrochloride. In the table below is listed the amount of actual glucosamine in the sulphated form or the hydrochloride form. A patient who consumes two 500 mg capsules of glucosamine sulphate is actually only getting a total of 650 mg of glucosamine. Similarly, if the patient consumes two 500 mg capsules of glucosamine hydrochloride, the patient is actually only getting a total of 830 mg of glucosamine. This can be further complicated by the level of purity of the glucosamine salt used in a given preparation. Assuming a purity of 90%, a patient in the above examples is actually only getting 580 mg or 740 mg of glucosamine, respectively. When comparing clinical trials, it is important to distinguish between the different salts because this determines the amount of the daily dose of glucosamine that the patient actually receives.

| | | Formula | Percent |
|---|---|---|---|
| Name of Substance | Molecular Formula | Weight | Glucosamine |
| Glucosamine | C₆H₁₃NO₅ | 179 | 100% |
| Glucosamine Sulphate | C₆H₁₃NO₅.H₂SO₄ | 277 | 65% |
| Glucosamine Chloride | C₆H₁₃NO₅.HCL | 215 | 83% |

Choosing a Glucosamine

Glucosamine comes in a number of different preparations, all of which have demonstrated beneficial effects. There is evidence that glucosamine acts only as a precursor and that the active inflammatory agent and structural agent is N-acetylglucosamine. Theoretically, in the clinical setting, the administration of N-acetylglucosamine rather than glucosamine would be preferred. The two most important reasons are 1) glucosamine is a precursor to the more biologically important N-acetylglucosamine and 2) N-acetylglucosamine has greater anti-inflammatory potency than glucosamine. The differences are itemized in the table below.

| Glucosamine Type | Positive Features | Negative Features |
|---|---|---|
| N-Acetylglucosamine | 1) The major constituent | 1) More expensive to |
| | of Hyaluronic Acid, a | produce than |
| | joint lubricant and | glucosamine salts |
| | shock absorber. | |
| | 2) Precursor of N- | |
| | Acetylgalactosamine, a | |
| | major constituent of | |
| | chondroitin sulphate | |
| | found in cartilage and | |
| | tendons. | |
| | 3) Direct anti- | |
| | inflammatory activity at | |
| | doses used clinically. | |
| Glucosamine | 1) Precursor of N- | 1) No direct anti- |
| (as a hydrochloride | Acetylglucosamine. | inflammatory activity |
| or sulphate salt) | 2) Less expensive to | at doses used |
| | produce than N- | clinically |
| | acetylglucosamine. | 2) Large percentage |
| | | of the administered |
| | | dose is the salt |

Glucosamine and N-acetylglucosamine are widely available in tablet and capsule form. Although a wide variety of doses of glucosamine sulphate have been used, better results occur with higher doses of three to six grams per day. Since most commercial preparations contain 500 mg of glucosamine sulphate per capsule, a dosage of three to six grams would require swallowing 6 to 12 capsules per day.

Liquid preparations of glucosamine and N-acetylglucosamine do not appear to be available to the general public, although clinical papers have reported the use of a glucosamine solution for injection and oral solution of N-acetylglucosamine as a therapy. Although glucosamine and N-acetylglucosamine have been administered through IV and IM routes, injections require needles and are not a desirable mode of delivery for most people An injection solution would need to be sterile and would have a limited shelf life.

Given that the ingestion of 6 to 12 capsules a day is onerous, the inventor queried whether a preferable form of delivery would be oral ingestion of glucosamine or N-acetylglucosamine in solution. Other than swallowing several capsules daily, a pleasant, painless mode of delivery of the glucosamine or N-acetylglucosamine that would encourage compliance was not available.

Two grams of glucosamine sulphate in 180 mL of water gives a solution with a pH of 6.06 and glucosamine hydrochloride gives a solution with a pH of 6.07. The solutions smell slightly fishy and the taste is unpalatable described as salty, sour to bitter.

A solution of 2 grams of N-acetylglucosamine in 180 mL of water has a pH of 7.40. This solution has no smell and a slightly sweetish taste.

Glucosamine Characteristics

| Type | Taste | pH (1% in water) |
|---|---|---|
| Glucosamine sulphate | Fishy and unpalatable | 6.06 |
| Glucosamine hydrochloride | Salty, sour to very bitter | 6.07 |
| N-Acetylglucosamine | Slightly sweet | 7.4 |

Glucosamine sulphate and glucosamine hydrochloride oral solutions are not palatable and would not provide a preferable mode of delivery. Although N-acetylglucosamine has a slightly sweetish taste, it is not so tasty that people would necessarily prefer it to swallowing capsules.

However, given the N-acetylglucosamine is not unpalatable and has a slightly sweetish taste, the inventor discovered that it could be incorporated into foods and beverages either before or after preparation. Although glucosamine alpha form has a melting point of 88° C. and the beta form decomposes at 110 degrees, N-acetylglucosamine has a melting point of 205° C. (16).

N-acetylglucosamine because of its higher melting point and its sweetish taste is ideal for making oral preparations that do not require taste masking. N-acetylglucosamine is stable in low temperature cooking and in hot liquids.

Since N-acetylglucosamine has a slightly sweetish taste, it can be added to liquids or foods that have a sweet taste or benefit from sweetening or are not detrimentally or noticeably affected by sweetening. N-acetylglucosamine could also be mixed with sugar or sugar substitutes in such a way that a desirable daily dosage would be easily consumed.

### EXAMPLES

Sweetener

Commonly used sweeteners, such as sugar, saccharin, aspartame, cyclamate, acesulfame K, sucralose, alitame, neotame and the like are often packaged in packets. The addition of a quantity of N-acetylglucosamine to such packets or a cube allows the consumer to obtain their desired daily dose of N-acetylglucosamine. For example, a packet of sugar containing 1, 2, 4 or 6 grams of N-acetylglucosamine can deliver the desired daily dosage when added to a food or a beverage. A packet of sugar containing N-acetylglucosamine is used in ordinary fashion, such as added to a hot or iced beverage like tea or coffee or sprinkled on a breakfast cereal.

Alternatively, N-acetylglucosamine may be used as a powder like sugar or packaged on its own in various quantities, such as 1, 2, 4 or 6 grams and these packets may be added to beverages and foods (during or after preparation) to deliver the desired amount of N-acetylglucosamine per serving size. The N-acetylglucosamine could also be in liquid form to be added to beverages and foods (during or after preparation), for example in a squirt or squeeze bottle.

The addition of 2 grams of N-acetylglucosamine to a cup of hot black tea, with or without the addition of milk, provides a tasty hot beverage. Likewise the addition of 2 grams of N-acetylglucosamine to a cup of hot black coffee, with or without the addition of cream or milk, provides a tasty hot beverage.

N-acetylglucosamine alone or with another sweetener may be added to recipes for baked goods. For example, a recipe for 12 muffins could deliver the desired quantity of 3 grams of N-acetylglucosamine per muffin if 36 grams of N-acetylglucosamine is added. To compensate for the fact that N-acetylglucosamine in powder or liquid is being added to a recipe, slightly more wet or dry ingredient can be added, respectively.

A desired quantity of N-acetylglucosamine may be added to single serving sizes of dessert, or snack to deliver the desired daily dosage. For instance, a packaged single serving of pudding, gelatin, cookies or dried fruit could include or be coated with between 3 and 6 grams of N-acetylglucosamine.

Beverage

A cold or hot beverage containing N-acetylglucosamine along with additional sweetening agents and flavouring agents, such as, orange, lemon or chocolate, can deliver the desired daily dosage. For example, if the amount of N-acetylglucosamine in a single serving of the beverage is 3 grams of N-acetylglucosamine, the consumer can have one or two drinks a day. Serving sizes could contain various other amounts of N-acetylglucosamine. The amount of a single serving size may be specified or be provided in a single bottle or can. Such beverage can also be in powder form to which water mild juice etc. is added.

A carbonated beverage containing N-acetylglucosamine along with additional sweetening agents and flavouring agents, such as cola, in a bottle, can or other suitable container, can deliver the desired daily dosage. Another example is a carton of chocolate milk containing the desired daily dosage of N-acetylglucosamine.

Medicinal Beverage Mix

A hot or cold beverage liquid or powder mix containing N-acetylglucosamine along with additional sweetening agents and/or flavouring agents and one or more medicinal agents, can deliver the desired daily dosage. Such medicinal agents include those that alleviate cold, flu and fever symptoms, such as an analgesic, decongestant, antihistamine, cough suppressant, and/or anti-inflammatory. This beverage can be packaged in single serving sizes such as a bottle or can, or packets of the powder mix.

Health Beverage

A hot or cold beverage liquid or powder mix containing N-acetylglucosamine, one or more additional sweetening agents and/or flavouring agents and one or more vitamins, minerals or herbs, can deliver the desired daily dosage. In addition to delivering the desired daily amount of N-acetylglucosamine, this beverage provides a specific amount of vitamins, minerals and/or herbs. For example, a cold beverage mix containing 3 grams of N-acetylglucosamine and the daily recommended dosage of vitamins. The added vitamin, mineral or herb are preferably water-soluble. This beverage can be packaged in single serving sizes such as a bottle or can, or packets of the powder mix.

Food

Cold breakfast cereal coated with N-acetylglucosamine with or without additional sweetening or flavoring agents can deliver the desired daily dosage in a regular single serving.

Hot breakfast cereal mixes including N-acetylglucosamine as a sweetener, with or without additional sweetening agents, can provide the required daily amount of N-acetylglucosamine in a single serving.

The desired daily dosage of N-acetylglucosamine can be added to soups, muffins, bread, banana bread, a cookie, dried fruit, etc. N-acetylglucosamine can be added during preparation (e.g. while preparing bread with the flour portion, or while cooking soup) or by sprinkling on top (e.g. cereal) or mixing in (e.g. a bowl of soup) suitable prepared foods.

Nutritional Bar

A nutritional bar containing between 3 and 6 grams of N-acetylglucosamine can deliver the desired daily dosage. A meal-replacement bar containing between 3 and 6 grams of N-acetylglucosamine can deliver the desired daily dosage.

Rehydration Solution

A rehydration solution containing between 3 and 6 grams of N-acetylglucosamine per serving size, can deliver the desired daily dosage.

Tea or Coffee

A packet of both tea leaves and N-acetylglucosamine, to be added to hot water, can deliver the desired daily dosage. A packet of instant coffee crystals containing N-acetylglucosamine, to be added to hot water, can deliver the desired daily dosage. The amount of N-acetylglucosamine in the packet of tea leaves or a serving of instant coffee could be 2, 3 or 6 grams.

In all of the above examples, consumption of more than the required daily dosage of N-acetylglucosamine (e.g. three cups of coffee) will not be harmful at the amounts tested clinically. In addition, consumption of N-acetylglucosamine is not restricted to provide those consumers with specific ailments, but rather is beneficial for the general population for relief of aches and pains. However, a beverage or food comprising a therapeutic amount of N-acetylglucosamine consumed daily can alleviate pain and inflammation in a patient suffering from inflammatory bowel disease or osteoarthritis.

### COMPARATIVE EXAMPLES

I. N-Acetylglucosamiiie Taste Test

Purpose: To test the sweetness of glucosamine sulphate and N-acetylglucosamine alone and in combination with another artificial sweetener.

Procedure:

The taste panel was asked to taste a small quantity of the various formulae and indicate the degree of sweetness, whether or not they like the taste and if there were any negative tastes. Sweetness and “like the taste” were scored using a 100 millimeter scale. Scores closer to 100 indicated high degree of sweetness or that the taste panel member very much “liked the taste”. The scores were averaged.

Glucosamine Formulae

N-Acetylglucosamine/Aspartame combinations

Per Gram of Mix

| Code | N-Acetylglucosamine (grams) | Aspartame |
|---|---|---|
| A1 | 0.985 | 0.015 |
| A2 | 0.9775 | 0.0225 |
| A3 | 0.955 | 0.045 |
| A4 | 0.94 | 0.06 |
| A5 | 1.00 | 0 |

Glucosamine sulphate Code A6

Results:

A total of 10 subjects participated in the taste test. All 10 subjects tested each of the formula A1 to A5. Three subjects taste tested formula A6.

Average Sweetness and “Like the Taste” Scores for the Various Formulae

| Formula | A1 | A2 | A3 | A4 | A5 | A6 |
|---|---|---|---|---|---|---|
| Sweetness Score | 57.2 | 59.1 | 71.5 | 69.6 | 35.6 | 8.7 |
| (1 to 100) | | | | | | |
| Like the Taste | 56.8 | 47.9 | 55.6 | 41.7 | 52.8 | 3 |
| (1 to 100) | | | | | | |
| N-Acetylglucosamine/ | 985/15 | 978/22 | 955/45 | 940/60 | 1/0 | 0/0 |
| Aspartame | | | | | | |
| Glucosamine Sulphate | 0 | 0 | 0 | 0 | 0 | 1/0 |

N-acetylglucosamine was less sweet than the combination of N-acetylglucosamine and aspartame but better liked than the combination formula with the highest concentration of aspartame. Descriptive words associated with the 100% N-acetylglucosamine formula included tastes like sugar, only slightly sweet, bland and not unpleasant. Only two subjects did not like the pure N-acetylglucosamine (formula A5). Almost all of the subjects described the texture of N-acetylglucosamine as smooth.

N-acetylglucosamine scored much sweeter than glucosamine sulphate and was much better liked than glucosamine sulphate. The words associated with glucosamine sulphate were bitter, salty, gritty, not palatable and awful.

Conclusions:

N-acetylglucosamine is a mildly sweet and palatable substance. The analog glucosamine sulphate, on the other hand, is unpalatable.

II. The Chocolate Taste Test

Purpose:

To test whether the addition of a therapeutically acceptable amount of glucosamine to a chocolate bar will negatively affect the taste and palatability of the chocolate bar.

Procedure:

Ingredients:

4-100 gram Cadbury milk chocolate bars with the following ingredients: sugar, milk cocoa butter, unsweetened chocolate, soya lecithin, natural and artificial flavours.

2 gram of N-acetylglucosamine Ferro-Pfanstiehl Lot #268948

2 grams of Glucosamine hydrochloride Wiler Lot #82634

2 grams of Glucosamine sulphate Wiler Lot #13338

In a double boiler melt the milk chocolate and add 2 grams of one of the glucosamine preparations indicated above. Mix thoroughly. For the control chocolate bar melt the chocolate, stir but do not add any ingredients. Pour out on sheet of aluminum foil and let the chocolate cool.

A taste panel took a piece of chocolate from each of the four chocolate formulae and were asked to record taste, after taste, and to indicate if they liked the taste or found it objectionable. Each taste panelist was asked to rank the 4 formula in order of preference where 1 indicated most liked and 4 least liked.

Results:

| Formula No. | Contents |
|---|---|
| 1 | Milk chocolate only |
| 2 | Glucosamine hydrochloride + milk chocolate |
| 3 | Glucosamine sulphate + milk chocolate |
| 4 | N-Acetylglucosamine + milk chocolate |

A total of fifteen panelist participated in the taste test of the 4 chocolate formulae.

Distribution of Ranking of Formulae in First, Second, Third, Fourth, or No Preference

| | Number (%) of Panelist Ranking Each Formula | | | | |
|---|---|---|---|---|---|
| Formula No. | First | Second | Third | Fourth | No Preference |
| 1 | 9 (60.0) | 1 (6.7) | 3 (20.0) | 0 (0) | 2 (13.3) |
| 2 | 1 (6.7) | 3 (20.0) | 5 (33.3) | 4 (26.7) | 2 (13.3) |
| 3 | 2 (13.3) | 2 (13.3) | 4 (25.7) | 6 (40) | 1 (6.7) |
| 4 | 6 (40.0) | 5 (33.3) | 0 (0) | 2 (13.3) | 2 (13.3) |

The numbers in any one column may be greater than 15 because some panelists ranked two formula equally.

Distribution of Panelist Who Liked or Did Not Like a Formula

| Formula No. | No. (%) Who Liked | | No. (%) Who Did Not Like | |
|---|---|---|---|---|
| 1 | 12 | (31.6) | 1 | (8.3) |
| 2 | 9 | (23.7) | 3 | (25.0) |
| 3 | 6 | (15.8) | 6 | (50.0) |
| 4 | 11 | (28.9) | 2 | (16.7) |

Number of Times (%) Certain Flavours Were Ascribed to Formulae

| Formula No. | Sweet | | Sour | Bitter | | Grainy/Gritty |
|---|---|---|---|---|---|---|
| 1 | 4 | (23.5) | 0 | 1 | (11.1) | 0 |
| 2 | 4 | (23.5) | 3 (100) | 2 | (22.2) | 0 |
| 3 | 2 | (11.8) | 0 | 6 | (66.7) | 7 (100) |
| 4 | 7 | (41.2) | 0 | 0 | (0) | 0 |

Conclusions:

1. The panelists ranked the control formula first 9 times and the N-acetylglucosamine formula first 6 times placing both formula well ahead of the glucosamine hydrochloride and glucosamine sulphate formulas. When the first and second ranking were combined, the control formula was chosen 10 times and the N-acetylglucosamine formula was chosen 11 times. The control and the N-acetylglucosamine containing formula placed well ahead of the glucosamine hydrochloride (4 times) and glucosamine sulphate (4 times).

2. Glucosamine hydrochloride and the glucosamine sulphate ranked fourth more frequently than either the control or the N-acetylglucosamine containing formula.

3. The control formula and the N-acetylglucosamine containing formula were liked more frequently than the glucosamine hydrochloride or glucosamine sulphate containing formula. The opposite was true when panelist expressed their dislike for a formula.

4. Panelists used the term sweet more frequently with the N-acetylglucosamine containing formula than any other formula. This indicates that N-acetylglucosamine acts as a sweetening agent when added to foods. This may also explain why some taste panelists found the N-acetylglucosamine containing formula too sweet and thus ranked this formula second.

5. The taste sour was ascribed to the glucosamine hydrochloride containing formula 3 times but not to the other formulae.

6. The taste bitter was ascribed once to the control formula, twice to the glucosamine hydrochloride containing formula and 6 times to the glucosamine sulphate containing formula.

7. Grainy and gritty were terms ascribed only to the glucosamine sulphate containing formula indicating that this substance does not dissolve completely in foods with a high lipid content.

8. N-acetylglucosamine when added to food has a pleasant and sweet taste. Other glucosamines such as glucosamine hydrochloride or glucosamine sulphate are associated with negative tastes when added to food.

III. N-Acetylglucosamine Compatibility with Foods and Beverages

Purpose:

To assess the impact on taste of N-acetylglucosamine when added to a wide variety of common foods and beverages.

Procedure:

Three formulae of N-acetylglucosamine were given to a five-member taste panel. The panel members were asked to add 1 teaspoon of N-acetylglucosamine to the foods and beverages they commonly use. They were then asked to record the formula's impact on taste and the taste acceptability of the altered taste if any.

| | Formula P | Formula Q | Formula R |
|---|---|---|---|
| Contents | (%) | (%) | (%) |
| N-Acetylglucosamine | 0.9825 | 0.9975 | 0.9525 |
| Aspartame | 0.015 | 0 | 0.045 |
| Cab-O-Sil | 0.0025 | 0.0025 | 0.0025 |

Cab-O-Sil is a fumed silica that acts as a flow agent.

In order to determine how much N-acetylglucosamine each subjected added to their food or beverage the weight of N-acetylglucosamine in a “level” teaspoon was determined.

Results:

The average weight of N-acetylglucosamine in a “level” teaspoon of N-acetylglucosamine is 1.3 grams.

The 5 panel members carried out a total of 73 taste tests with the following foods and beverages:

| | Formula P | Formula Q | Formula R |
|---|---|---|---|
| Foods | Cornflakes | All Bran | Apple |
| | Cream of Wheat | Banana | Cake |
| | Rice | Cream of Wheat | Cereal |
| | Yogurt | Grapefruit | Cream of Wheat |
| | | Shreddies | Grapefruit |
| | | Yogurt | |
| Bever- | Annise Milk | Chamomile Tea | Chamomile Tea |
| ages | (hot) | (hot) | (hot) |
| | Chamomile Tea | Chocolate Drink | Coffee |
| | (hot) | (hot) | (hot) |
| | Coffee with Milk | Coffee | Milk |
| | (hot) | (hot) | (hot) |
| | Lemonade (cold) | Milk (hot) | Lemonade (cold) |
| | Orange Juice (cold) | Skim Milk (hot) | Orange Juice (cold) |
| | Orange/Tangerine | Skim Milk (cold) | Tea (hot) |
| | Juice (cold) | Orange Juice (cold) | Tea (cold) |
| | Tea (hot) | Orange/Tangerine | Water |
| | Tea (cold) | Juice (cold) | |
| | | Tea (hot) | |
| | | Tea (cold) | |

Distribution of the number of times taste panellists expressed a like (Yes) or a dislike (No) for a given formula when added to various foods and beverages:

| | | | | |
|---|---|---|---|---|
| Formula | No. of | | | |
| # | Trials | No. of No (%) | No. of Yes (%) | No. of Unsure (%) |

| | | | | |
|---|---|---|---|---|
| P | 25 | 6 (24.0) | 15 (60.0) | 4 (16.0) |
| Q | 28 | 3 (10.7) | 19 (67.9) | 6 (21.4) |
| R | 20 | 10 (50.0) | 8 (40.0) | 2 (10.0) |

Reasons for not liking a formula:

| | No. | |
|---|---|---|
| Formula # | of No | Reasons |
| P | 6 | Too sweet (2), Changes taste (2), Bitter after taste (2) |
| Q | 3 | Not sweet enough (2), Taste bad in orange juice (1) |
| R | 10 | Too sweet (9), Bitter after taste (1) |

Conclusions:

1. N-acetylglucosamine is compatible with a wide variety of foods and drinks both hot and cold.

2. N-acetylglucosamine without aspartame imparts sweetness to these foods and beverages.

3. To achieve additional sweetness a very small amount of an artificial sweetener may be added.

The Muffin Taste Test

Purpose:

To test whether the addition of a therapeutically acceptable amount of glucosamine to a bran muffin mix will negatively affect the baking process, and the taste and palatability of the bran muffin.

Procedure:

Ingredients:

Quaker Bran Muffin Mix UPC 5557710487 Lot #0427P2 and Lot #0527P2 containing flour, cane sugar, wheat bran, vegetable oil, shortening, modified milk ingredients, salt, sodium bicarbonate, monocalcium phosphate, caramel colour and artificial flavour.

36 gram of N-acetylglucosamine Ferro-Pfanstiehl Lot #268948

36 grams of Glucosamine hydrochloride Wiler Lot #82634

36 grams of Glucosamine sulphate Wiler Lot #13338

Other ingredients: 2 eggs lightly beaten, water

Preparation of Control and Test Articles

Measure out 4 times 415 mL dry muffin mix (sufficient to make 6 muffins each) to make up the following formulae

| Formula # | Glucosamine content |
|---|---|
| A | Glucosamine sulphate 36 grams (6 grams per muffin) |
| B | N-acetylglucosamine (6 grams per muffin) |
| C | Nil - Control |
| D | Glucosamine hydrochloride (6 grams per muffin) |

Add to each dry mix 160 mL of water and/4 of egg mixture and mix thoroughly.

Pour equal amounts into 6 muffin cups for each of the formulae.

Preheat oven to 400° F. or 204° C., bake each formula separately and observing cooking behaviour.

A taste panel took a piece of muffin from each of the four muffin formulae and were asked to record taste, after taste, and to indicate if they liked the taste or found it objectionable. Each taste panellist was asked to rank the 4 formulae in order of preference where 1 indicated most liked and 4 least liked.

Results:

Baking:

| Formula # | Baking Results |
|---|---|
| A | After 15 minutes the top had very dark brown to burnt |
| | appearance - removed from oven |
| B | At 15 minutes, muffin had risen, At 18 minutes light brown |
| | muffin - removed from oven |
| C | At 15 minutes, muffin had risen, At 20 minutes light brown |
| | muffin - removed from oven |
| D | After 15 minutes the top very dark with burnt edges. |
| | At 17 minutes dark and burnt on top - taken out of oven. |
| | When muffins removed from paper cups, the paper was |
| | blackened and part of it had disintegrated. |

Muffins were cut up into bite size pieces and identified only as A, B, C, or D. A total of thirteen panellists participated in the taste test of the 4 muffin formulae.

Distribution of Ranking of Formulae in First, Second, Third, Fourth, or No Preference

| | Number (%) of Panelist Ranking Each Formula | | | | |
|---|---|---|---|---|---|
| Formula No. | First | Second | Third | Fourth | No Preference |
| A | 0 (0) | 0 (0) | 5 (38.5) | 8 (61.5) | 0 (0) |
| B | 9 (69.2) | 4 (30.8) | 0 (0) | 0 (0) | 0 (0) |
| C | 6 (46.2) | 7 (53.8) | 0 (0) | 0 (0) | 0 (0) |
| D | 0 (0) | 0 (0)) | 3 (23.1) | 10 (76.9) | 0 (0) |

The numbers in any one column may be greater than 13 because some panellists ranked two formula equally.

Distribution of Panellists Who Liked or Did Not Like a Formula

| Formula No. | No. (%) Who Liked | No. (%) Who Did Not Like |
|---|---|---|
| A | 0 (0) | 13 (46.4) |
| B | 13 (54.2) | 0 (0) |
| C | 11 (45.8) | 2 (7.1) |
| D | 0 (0) | 13 (46.4) |

Number of Times (%) Certain Flavours Were Ascribed to Formulae

| Formula No. | Sweet | Sour | Bitter | Grainy/Gritty |
|---|---|---|---|---|
| A | 1 (7.7) | 6 (42.9 | 5 (62.5) | 0 (0) |
| B | 7 (53.8) | 0 (0) | 0 (0) | 0 (0) |
| C | 5 (38.5) | 0 (0) | 0 (0) | 0 (0) |
| D | 0 (0) | 0 | 3 (37.5) | 0 (0) |

Discussion and Conclusions:

Bran muffins were baked at 400° F. (204° C.). Glucosamine hydrochloride and Glucosamine sulphate containing bran muffins darkened and took on a burnt appearance on the outside. In contrast the N-acetylglucosamine containing muffin exhibited a pale golden colour similar to the control. The physical properties of the analogs help explain this observation. Glucosamine has a melting point of 88° C. and decomposes at 110° C. N-acetylglucosamine has a melting point of 205° C. Since the baking temperature of bran muffins is 204° C., glucosamine hydrochloride and glucosamine sulphate break down in the baking process. In contrast N-acetylglucosamine with its much higher melting point is stable during the baking process.

The panelists ranked formula C (control) first 6 times and the N-acetylglucosamine containing formula (B) first 9 times placing both formula well ahead of the glucosamine hydrochloride and glucosamine sulphate formulas. When the first and second ranking were combined, the control formula was chosen 13 times and the N-acetylglucosamine formula was chosen 13 times. The control and the N-acetylglucosamine containing formula placed well ahead of the glucosamine hydrochloride (0 times) and glucosamine sulphate (0 times). Clearly N-acetylglucosamine is an acceptable addition to a baked food.

Glucosamine hydrochloride and the glucosamine sulphate formula ranked third and fourth. Neither the control nor the N-acetylglucosamine containing formula was ranked third or fourth by any of the panellists.

The control formula and the N-acetylglucosamine containing formula were universally liked. Neither formula received a negative rating. Both the glucosamine hydrochloride or glucosamine sulphate containing formula received a negative rating but never a positive rating.

Panellists used the term sweet more frequently with the N-acetylglucosamine containing formula (7 times) than any other formula. The term sweet was used 5 times in conjunction with the control formula. The term sweet was used once in conjunction with the glucosamine sulphate formula but never with the glucosamine hydrochloride containing formula. This confirms that N-acetylglucosamine acts as a sweetening agent when added to foods.

The taste sour/salty was ascribed to the glucosamine hydrochloride containing formula 8 times and to the glucosamine sulphate formula 6 times. This negative attribute was never associated with the control or the N-acetylglucosamine containing formula.

The taste bitter was ascribed 5 times to the glucosamine sulphate containing formula and 3 times to the glucosamine hydrochloride containing formula. This negative attribute was never associated with the control or the N-acetylglucosamine containing formula.

N-acetylglucosamine when added to food for baking has a pleasant and sweet taste. Other glucosamines such as glucosamine hydrochloride and glucosamine sulphate are associated with negative tastes when added to food and break down during the heating process.

### BIBLIOGRAPHY

1. Textbook of Biochemistry with clinical correlations, Fourth edition. 1997 Thomas M Devlin editor Wiley-Liss Inc. Publisher Toronto

2. Burton, A. F. and F. H. Anderson (1983). “Decreased incorporation of 14C-glucosamine relative to 3H—N-acetyl glucosamine in the intestinal mucosa of patients with inflammatory bowel disease.” Am J Gastroenterol 78(1): 19-22.

3. Shikhman, A. R., K. Kuhn, et al. (2001). “N-acetylglucosamine prevents IL-1 beta-mediated activation of human chondrocytes.” J Immunol 166(8): 5155-60.

4. McCarty, M. F. (1998). “Enhanced synovial production of hyaluronic acid may explain rapid clinical response to high-dose glucosamine in osteoarthritis.” Med Hypotheses 50(6): 507-10.

5. Reginster, J. Y., R. Deroisy, et al. (2001). “Long-term effects of glucosamine sulphate on osteoarthritis progression: a randomised, placebo-controlled clinical trial.” Lancet 357(9252): 251-6.

6. Qiu, G. X., S. N. Gao, et al. (1998). “Efficacy and safety of glucosamine sulfate versus ibuprofen in patients with knee osteoarthritis.” Arzneimittelforschung 48(5): 469-74.

7. Das, A., Jr. and T. A. Hammad (2000). “Efficacy of a combination of FCHG49 glucosamine hydrochloride, TRH122 low molecular weight sodium chondroitin sulfate and manganese ascorbate in the management of knee osteoarthritis.” Osteoarthritis Cartilage 8(5): 343-50.

8. Noack, W., M. Fischer, et al. (1994). “Glucosamine sulfate in osteoarthritis of the knee.” Osteoarthritis Cartilage 2(1): 51-9.

9. Rindone, J. P., D. Hiller, et al. (2000). “Randomized, controlled trial of glucosamine for treating osteoarthritis of the knee.” West J Med 172(2): 91-4.

10. Houpt, J. B., R. McMillan, et al. (1999). “Effect of glucosamine hydrochloride in the treatment of pain of osteoarthritis of the knee.” J Rheumatol 26(11): 2423-30.

11. Thie, N. M., N. G. Prasad, et al. (2001). “Evaluation of glucosamine sulfate compared to ibuprofen for the treatment of temporomandibular joint osteoarthritis: a randomized double blind controlled 3 month clinical trial.” J Rheumatol 28(6): 1347-55.

12. Salvatore, S., R. Heuschkel, et al. (2000). “A pilot study of N-acetyl glucosamine, a nutritional substrate for glycosaminoglycan synthesis, in paediatric chronic inflammatory bowel disease.” Aliment Pharmacol Ther 14(12): 1567-79.

13. Levin, R. M., N. N. Krieger, et al. (1961). “Glucosamine and N-Acetylglucosamine.” J. Lab & Clin Med 58: 927-932.

14. Setnikar, I. and L. C. Rovati (2001). “Absorption, distribution, metabolism and excretion of glucosamine sulfate. A review.” Arzneimittelforschung 51(9): 699-725.

15. Pouwels, M. J., J. R. Jacobs, et al. (2001). “Short-term glucosamine infusion does not affect insulin sensitivity in humans.” J Clin Endocrinol Metab 86(5): 2099-103.

16. Entry 4352; Merck Index 11^{th} Edition Published by Merck & Co., Inc. Rahway, N.J., USA.

The embodiments of the invention in which an exclusive property or privilege is claimed are defined as follows:

1. The use of N-acetylglucosamine as an additive to foods and beverages wherein each single serving size of said food and beverage comprises from about 0.1 grams to 20 grams of N-acetylglucosamine.

2. The use of N-acetylglucosamine as a sweetener of foods and beverages wherein each single serving size of food and beverage comprises from about 0.1 grams to 20 grams of N-acetylglucosamine.

3. The use in claims 1 or 2, wherein each single serving size of food and beverage comprises from about 1 gram to 6 grams of N-acetylglucosamine.

4. The use in claims 1 or 2, wherein said beverage is chosen from the following list: cola, iced tea, hot tea, iced coffee, hot coffee, juice, hot milk, cold milk, cold chocolate milk, and hot chocolate.

5. A hot or cold beverage composition comprising from about 0.1 grams to 20 grams of N-acetylglucosamine per single serving size in a beverage.

6. The beverage composition of claim 5, wherein said beverage is chosen from the following list: cola, iced tea, hot tea, iced coffee, hot coffee, juice, hot milk, cold milk, cold chocolate milk, and hot chocolate.

7. A drink mix composition comprising from about 0.1 grams to 20 grams of N-acetylglucosamine per single serving size in a drink mix.

8. The drink mix composition of claim 7, wherein the drink mix is one of the following: a bag of tea leaves, instant coffee crystals, flavoured crystals.

9. The drink mix composition of claim 7, wherein the drink mix is a powder reconstituted by addition of one or more of water, milk or juice.

10. The use in claims 1 or 2, wherein said food is chosen from the following list: cookies, muffins, banana bread, cereal, soup, dried fruit, bread.

11. A hot or cold food composition comprising from about 0.1 grams to 20 grams of N-acetylglucosamine per single serving size in a food.

12. The food composition of claim 11, wherein said food is chosen from the following list: cookies, muffins, banana bread, cereal, soup, dried fruit, bread.

13. A nutritional bar composition comprising 0.1 grams to 20 grams of N-acetylglucosamine per single serving size in a nutritional bar.

14. A meal replacement bar composition comprising 0.1 grams to 20 grams of N-acetylglucosamine per single serving size in a meal replacement bar.

15. A medicinal drink in liquid or powder form comprising 0.1 grams to 20 grams of N-acetylglucosamine and a pharmaceutically acceptable amount of medicine per single serving size.

16. The medicinal drink of claim 15 wherein the medicine is one or more substances chosen from the following list: analgesic, decongestant, antihistamine, cough suppressant, anti-inflammatory.

17. A health beverage or food comprising 0.1 grams to 20 grams of N-acetylglucosamine and a beneficial daily amount of vitamins, minerals and/or herbs, per single serving size.

18. A rehydration solution comprising 0.1 grams to 20 grams of N-acetylglucosamine per single serving size.

19. An edible composition for alleviating pain and/or inflammation in a patient suffering from inflammatory bowel disease or osteoarthritis, comprising a therapeutic amount of N-acetylglucosamine dissolved in a palatable liquid beverage.

20. An edible composition for alleviating pain and/or inflammation in a patient suffering from inflammatory bowel disease or osteoarthritis, comprising a therapeutic amount of N-acetylglucosamine in a food.

21. The use of the beverage composition of claim 5, or the drink mix composition of claim 7, or the food composition of claim 11, for alleviating pain and/or inflammation, in a patient suffering from osteoarthritis or inflammatory bowel disease.

22. The use of the beverage composition of claim 6, or the drink mix composition of claim 8 or claim 9, or the food composition of claim 12, for alleviating pain and/or inflammation, in a patient suffering from osteoarthritis or inflammatory bowel disease.

23. The use of claims 1 or 2, wherein the additive or sweetener is added during preparation of the food or beverage.

24. The use of claims 1 or 2, wherein the additive or sweetener is added after preparation of the food or beverage.

25. A sweetener comprising N-acetylglucosamine in powder form.

26. A sweetener comprising N-acetylglucosamine and one or more of the following: sugar, saccharin, aspartame, cyclamate, acesulfame K, sucralose, alitame, neotame.

27. The sweetener of claims 25 or 26, wherein the sweetener is packaged in bulk, single serving packets or cubes.

28. A sweetener comprising N-acetylglucosamine in liquid form.

29. The sweetener of claim 28, wherein the sweetener is packaged in a squirt or squeeze bottle.
